(19) United States
(12) Patent Application Publication
(10) Pub. No.: US 2010/0247503 A1
(43) Pub. Date: Sep. 30, 2010

(54) METHOD FOR OBTAINING A SOLID COMPOSITION WITH RHODOTORULA MINUTA, WHICH IS EFFECTIVE FOR THE BIOLOGICAL CONTROL OF ANTHRACNOSE, AND RESULTING COMPOSITION

(76) Inventors: Enrique Galindo Fentanes, Cuernavaca (MX); Leobardo Serrano Carreon, Cuernavaca (MX); Jose Armando Carrillo Fasio, Culiacan (MX); Raul Allende Molar, Culiacan (MX); Raymundo Saul Garcia Estrada, Culiacan (MX); Lizette Trujillo Robles, Tuxtia (MX); Martin Patino Vera, Yautepec (MX)

Correspondence Address: JACOBSON HOLMAN PLLC, 400 SEVENTH STREET N.W., SUITE 600, WASHINGTON, DC, 20004, US

(21) Appl. No.: 12/311,802

(22) PCT Filed: Oct. 16, 2006
(86) PCT No.: PCT/MX2006/000108

§ 371 Date: May 28, 2010

(51) Int. Cl. A01N 63/04; C12N 1/16; A01N 63/00; A01P 3/00

(52) U.S. Cl. 424/93.462; 435/255.1; 424/93.51

(57) ABSTRACT

The invention relates to a method for producing a dry solid composition which is effective in the biological control of Colletotrichum gloeosporioides, contains Rhodotorula minuta and has a shelf life of at least one year. The invention also relates to the resulting composition and to a method for using same as a biological control agent. The invention further relates to a dry solid composition containing Rhodotorula minuta with a shelf life of up to one year when refrigerated and a second biological control agent, Bacillus subtilis, which can be used to obtain anthracnose severity control levels equal to or greater than the levels achieved with larger doses of those used with the same control agents, when applied independently. Finally the invention describes a method for reducing weight loss during the post-harvest storage of mango.

### TECHNICAL FIELD

This invention relates to methods for obtaining solid compositions with Rhodotorula minuta, a biological control agent effective against Colloetotrichum gloeosporioides, the cause of the main fungal disease of the mango. These methods allow obtaining an effective biofungicide with a long shelf life. These compositions may comprise, in addition to Rhodotorula minuta, another biological control agent, such as Bacillus subtilis, to reduce the dose of the control agents. It also refers to the pre-harvest application of such compositions for the biological control of the fungal phytopathogen Colletotrichum gloeosporioides, without negatively affecting the main parameters of the quality of the fruit. Furthermore, this invention also relates to a method for reducing mango loss of weight during storage, characterized in that it is applied in at least one pre-harvest application of a solid composition, dry, with Rhodotorula minuta, effective for biological control of Colletotrichum gloeosporioides.

### BACKGROUND OF THE INVENTION

Fungal diseases of the mango, such as anthracnose, can reduce fruit quality and cause post-harvest losses of up to 60% of total production (Vega, 2001). In extreme cases, a high incidence of this disease can cause losses of up to 100% of the fruit produced in climates with high humidity (Arauz, 2000). This disease is caused by the phytopathogen fungus Colletotrichum gloeosporioides Penz (Freeman et al., 1998). Anthracnose of the mango has a very high incidence in almost all areas producing this fruit. The pathogenic fungus usually infects the mango tree early in the production cycle. Consequently, the disease must be primarily controlled prior to affecting the productivity of the tree and, although symptoms may not be apparent at the time of picking of the fruit, they may be manifested throughout post-harvest handling. While many producers carry out post-harvest fungicide chemical treatments, pre-harvest control of anthracnose determines the final quality of the fruit (Arauz, 2000).

The biological control of plant diseases is a viable alternative to chemical control of phytopathogens. This involves the use of microorganisms (usually from the same habitat as the pathogen) that inhibit or prevent the development of the pathogenic microorganism. These microorganisms have high specificity and environmental safety (Spadaro and Gullino, 2004). Among the biological antagonists there are bacteria and yeasts, the latter because they generally tolerate many of the fungicides and chemicals used pre and/or post-harvest (Spadaro and Gullino, 2004), which allows producers to use them in combination with low toxicity chemical fungicides.

The yeasts of the Rhodotorula genus (in particular Rhodotorula glutinis) have been reported as good biological control agents (Shanmuganathan, 1996; Chand-Goyal and Spotts, 1998) against Penicillum expansum in apples and pears (Benbow and Sugar, 1999) and against Botrytis cinerea in strawberries when applied pre and/or post-harvest (Helbig, 2001). Rhodotorula species are generally considered safe for humans. Naidu et al. (1999) demonstrated that oral feeding of frozen cells, from 0.5 to 6.0 g/Kg. body weight, of this yeast, did not produce toxic effects in male and female albino rats. Specifically for Rhodotorula minuta, the American Type Culture Collection (ATCC, www.atcc.org) has classified it Biosafety Level 1, meaning that it is not recognized as the cause of diseases affecting the health of human adults.

Biological control of fungal diseases has been reported in other fruit, including apples, avocado, papaya and mango. For diseases caused in apples by Penicillium spp. Botrytis spp., Mucor spp., Pezicula spp., Phialophora and/or Monilinea spp., it is recommended to use mixes of: Cryptococcus infirmo-miniatus, Cryptococcus laurentii, Rhodotorula aurantiaca and Rhodotorula glutinis (Chand-Goyal and Spotts, 1998). For diseases caused by Pseudocercospora purpurea in avocados it is recommended to use Bacillus subtilis (Korsten et al., 1997). For diseases caused by C. gloeosporioides, in papaya the use of Candida oleophila is recommended (Gamagae et al., 2004), and, in regard to anthracnose in mango, De Jager et al. (2001) investigated the application of Bacillus spp., finding that latent infections could be controlled. Other successful cases in mango include the post-harvest application of Pseudomonas fluorences, reported by Koomen and Jeffries (1993). However, semi-commercial or commercial trials have not been reported in any of the above cases. Only the authors of this invention have reported the yeast Rhodotorula minuta as a biological control agent.

Other authors have reported the production of Rhodotorula minuta for other purposes, for example, to produce and extract pigments (β-carotene). Velankar and Heble (2003) reported that R. minuta can grow at pH values between 4 and 9 and temperatures between 20 and 30° C., grown in different liquid mediums.

Carrillo-Fasio et al. (2005) reported the Bacillus subtilis bacteria and the yeast Rhodotorula minuta as biological control agents effective against anthracnose of the mango caused by the fungal phytopathogen Colletotrichum gloeosporioides Penz. The bacteria presented with the phenomenon of antagonism by antibiosis and it was found that the yeast Rhodotorula minuta primarily presented with the phenomenon of competition (Patiño-Vera et al., 2005). Strains of Bacillus subtilis and Rhodotorula minuta are available in internationally recognized microorganism deposits.

The inventors of this invention reported (Patiño-Vera et al., 2005) biological control of anthracnose in mango, in semi-commercial field trials in three production seasons (indispensable prerequisite for corroborating effectiveness) through applications of liquid formulations of the yeast Rhodotorula minuta in pre-harvest. To produce enough viable yeast for such semi-commercial applications, the production process was successfully escalated by submerged fermentation from a laboratory flask to a 100 L bioreactor, using a low-cost culture medium, being the first to report an escalation process to obtain viable cells of R. minuta in high concentrations (up to 2×10⁹ CFU/mL). The viable cells obtained in the pilot fermenter exceeded the values obtained in the stirred flasks and were achieved in less time. The yeasts were harvested in the exponential growth stage.

However, all formulations tested until then presented a major technical problem: lack of sufficient refrigeration shelf life for commercialization. The viability of the yeasts declined rapidly in the first two months of storage. The minimum shelf life of 5 to 6 months is recommended (Janisiewicz and Korsten, 2002), to be consistent with the practices of routine handling and storage in the agrochemical market. Formulating the yeast at a concentration of 1×10⁹ CFU/mL and adding glycerol (20%) and xanthan gum (5 g/L) it was possible to avoid bacterial contamination and cellular sedimentation, being able to preserve up to an order of 10⁷ CFU/mL for six months (Patiño-Vera et al., 2005). This can be an insufficient cell concentration, considering that doses of up to 10⁸ CFU/mL of R. minuta may be needed to achieve anthracnose control results similar or better than those achieved when using chemical fungicides such as Benomyl. The problem is exacerbated because the loss of viability increases when increasing R. minuta cell concentration. A liquid composition with an initial concentration of 10¹⁰ CFU/mL of R. minuta, and a pH phosphate buffer, caused a dramatic decrease in the concentration of living cells for up to five orders of magnitude after one month of refrigerated storage, compared with that initially formulated with 10⁹ CFU/mL of yeast (Patino-Vera et al., 2005).

Therefore, it was essential to investigate alternatives for increasing the shelf life of the concentrated formula. In a practical sense, Janisiewicz and Jeffers (1997) have pointed out that the biggest hurdle in the commercialization of products for biological control is the development of formulations with a stable shelf life that would allow retaining an objective activity of controlling the severity of the target disease similar to that obtained with products made with fresh cells, in whose formulations the vast majority of the cells are alive.

Different strategies have been proposed to deal with the problems outlined above, but these have not adequately solved the problem when the control agents are yeasts or filamentous fungi. Other alternatives have used very expensive processes (such as lyophilization) to dry out formulations of biological control agents. For example, Abbey et al. (2001) reported that despite having used protective agents such as powdered skim milk, lactose, fructose, glucose, sucrose and/or various combinations thereof, in addition to different modes of rehydration, the effectiveness of freeze-dried cells of another yeast, Candida sake, was significantly lower than when the cells were not freeze-dried. Cheaper processes such as spray-drying have not been effective in preserving bacteria, conidial fungi or yeasts. For example, in the case of lactic acid bacteria spray-dried and stored under refrigeration, a decrease in their viability was reported after three months of storage (Wan-Yin and Mark, 1995). Jones et al. (2004) reported that, in general, the conidia of the biological control agent Coniothyrium minitans, spray dried, presented a germination percentage lower than those which were not dried. In the case of other biological control agents (Pantoea agglomerans), a recovery rate of about 50% after spray drying and a final low viability were reported (Costa et al. 2002). In recently reported trials, Abbey et al. (2005) indicated that in the case of spray-dried Candida sake yeast, this biological control agent is significantly less effective against fungal phytopathogens in apples than the fresh cell. These authors concluded that spray-drying is not a good method for the dehydration of yeasts because few of them survived (only 10%), poor product recovery that is especially ineffective for controlling the disease.

The feasibility of drying Rhodotorula type yeasts for other purposes has been previously reported, i.e. for producing and extracting β-carotenes using Rhodotorula glutinis. Bhosale et al. (2003) reported that the culture broth with this yeast can be concentrated up to 10 times by microfiltration followed by spray drying, resuspending concentrated yeast at concentrations of 1 to 15% (w/v), with input temperatures of 40 to 200° C., and output temperatures from 33 to 84° C. After the drying process, viable cells can be detected in an input temperature range of 40 to 180° C. and output temperatures from 33 to 82° C.

Abadias et al., (2005) used powdered skim milk, high-lactose powdered skim milk or magnesium sulphate as protective or carrier support in the drying of the biological control agent Candida sake. On the other hand, Lodato et al. (1999) report having used sugars and polymer matrixes (maltodextrins) as supports in dry lyophilization. These same authors reported that the addition of carbohydrates in the composition of the microorganism suspension before drying helps to preserve its viability and increase the shelf life of the dry product. This is possibly due to the ability of carbohydrates to establish hydrogen bridges that help stabilize vital proteins. They also report that the degree of protection is determined by the carbohydrate content of saccharides with low molecular weight. Carbohydrates with a higher content of saccharides with low molecular weight showed a greater degree of protection. On the other hand, the formulation of the biological control agent with supports or protectors can increase the recovery of dry powder and help to control its moisture content (Abadias et al., 2005).

Larena et al. (2003) demonstrated that in addition to spray drying, conidia of Penicillum oxalicum can be dried by lyophilization or fluidized bed drying. Using these two processes, 100% of their viability can be preserved, but it must be previously formulated with high-cost protective supports, such as powdered skim milk with Tween 20 or peptone milk. Without the addition of these supports or protectors, the conidia did not maintain their viability at room temperature, whereas the addition of protectors retained viability between 40 to 50% up to 180 days. With spray drying, the value of the viability decreased to 20%. Finally, they proved that the fluidized bed-dried conidia retained their effectiveness as biological control agents.

Consequently, alternative investigations are essential for the formulation and packaging of biological control agents, to allow for their adequate conservation for appropriate periods for marketing. On the other hand, several authors have reported inconsistency in the effectiveness of biological control agents when applied in commercial orchards (Janisiewicz and Jeffers, 1997; Janisiewicz and Korsten, 2002; Guetsky et al., 2001). These inconsistencies [can be overcome] and other benefits can be achieved by employing compatible mixtures of biological control agents (Janiciewicz and Jeffers, 1997; Janisiewicz and Korsten, 2002). Therefore, it is also necessary to have alternative mixes of compatible biological control agents to overcome these inconsistencies and obtain other benefits, such as reducing the dose of antagonist microorganisms required (Spadaro and Gullino, 2004) and thus contribute to limit the loss of viable cells during the formulation, concentration and/or drying processes.

### BRIEF DESCRIPTION OF THE FIGURES

FIG. 1. Graphic showing kinetics of growth and glucose consumption of Rhodotorula minuta yeast grown in a 10 L fermenter.

FIG. 2. The graphic shows the recovery efficiency of CFU and total solids in the spray drying process of Rhodotorula minuta from centrifuged and non-centrifuged fermentation broths.

FIG. 3. Comparison of “kinetic control” of the germination percentage of C. gloeosporioides without the presence of R. minuta and an example where a dry solid composition is added to a previously formulated R. minuta.

FIG. 4 The graphic shows a comparison of the antagonistic effect of solid and liquid formulations of Rhodotorula minuta, measured as the germination percentage of the conidia 56 h from co-culture.

FIG. 5. Graph showing the concentration of viable R. minuta cells in the solid and liquid formulations, along with the shelf-life.

FIG. 6. This graph shows the behavior of viable R. minuta cells during an accelerated aging treatment (stored at 37° C.) of an R. minuta formulation with support and a control.

### BRIEF DESCRIPTION OF THE INVENTION

This invention is based on previous research of the inventors, which led them to be the first in the world to report on the yeast Rhodotorula minuta as a biological control agent against mango anthracnose (Patiño-Vera et al., 2005) and on the substantial number of technical improvements that have been incorporated into the processes of production, formulation, drying and application of biological control agents (separately in combination) against C. gloeosporioides, the cause of anthracnose, especially in mangos. These improvements enabled them to establish the method of this invention, comprising a first stage consisting of a process of Rhodotorula minuta cell production, an optional second stage of recovery of the cell pack and its resuspension; an optional third stage of formulation; and a fourth stage of drying to obtain a solid composition with Rhodotorula minuta, effective for biological control of anthracnose caused by C. gloeosporioides.

The method of this invention provides a dry solid composition, effective as a biological control agent, with a long shelf life under refrigeration (over 6 months, as recommended by Janisiewicz and Korsten, 2002).

The invention also regards a composition comprising R. minuta and a second biological control agent effective against C. gloeosporioides, particularly Bacillus subtilis. The advantage of this composition is that it is effective in controlling the severity of anthracnose in lower doses than those required for the same control agents when applied separately.

The invention also includes a method for biological control of C. gloeosporioides, which includes at least one pre-harvest application of effective doses of a solid composition, dry, with R. minuta.

The invention also includes a method for reducing weight loss during mango storage, characterized by at least one pre-harvest application of a solid composition, dry, with R. minuta, effective for biological control of C. gloeosporioides.

The methods of this invention are useful for dry compositions, including R. minuta with a long shelf life and effective in controlling C. gloeosporioides, a pathogen of mango and other fruits, with an industrial-scale technology, which uses inexpensive raw materials, allowing for the use of antagonistic microorganisms, individually or in combinations. The latter form makes it possible to decrease the dose of biological control agents required to control the severity of the disease caused by the fungal pathogen. The organic products developed are easy to handle and apply, and have a shelf life of at least one year under refrigeration, which is very convenient and suitable for their eventual distribution and marketing in the agrochemical market.

### DETAILED DESCRIPTION OF THE INVENTION

### Definitions

The term “to culture” refers to the spreading of organisms on or in various types of solid and liquid culture mediums.

As used herein, “biological control of C. gloeosporioides” is defined as the act of reducing, limiting, mitigating, stabilizing, reversing, slowing or delaying the severity of disease caused by the fungal phytopathogen C. gloeosporioides, by applying one or more antagonistic microorganisms (biological control agents).

This invention indistinctively uses the terms antagonist or biological control agent to refer to a microorganism with biological control activity against C. gloeosporioides.

An “effective product” is a product which, when applied in sufficient quantity, achieves effective biological control of C. gloeosporioides. An effective product can be administered in one or more applications. In terms of treatment and protection, an “effective treatment” is the dose and number of applications sufficient to achieve an effect of biological control of C. gloeosporioides.

Tree, as defined herein, includes the largest portion of the plant, including shoots, the stem, nodes, internodes, petioles, leaves, flowers, fruits, etc.

This invention solves the technical problem of the low shelf life of liquid compositions of R. minuta reported so far, by a method for producing a solid, dry composition, effective in the biological control of Colletotrichum gloeosporioides, including Rhodotorula minuta and possessing a shelf life of at least one year.

The production of Rhodotorula yeasts has been previously reported for application in the biological control of plant diseases by submerged fermentation. However, most of these processes are carried out in laboratory flasks or fermenters. Moreover, the processes reported are designed to obtain viable cells in full growth (exponential stage), using bacteriological or laboratory grade culture mediums. Corroborating the effectiveness of biological control agents in the field requires semi-commercial quantities of such agents. To do so, it is necessary to escalate the production processes at least to semi-industrial production level, using low-cost culture mediums. There are no specific reports by other authors concerning the aspects of the production process of Rhodotorula minuta to obtain cells resistant to drying, in which the recovery yields of living cells (measured as CFU/mL) are sufficient to develop semi-commercial prototypes that retain, once dried, effective control of the severity of a fungal disease. In these last two aspects, the inventors of this invention have been the first to achieve this, because, as already reported in the antecedents, several authors have failed in their attempt to develop dry products with high concentrations of effective biological control agents (Wan-Yin and Mark, 1995, Jones et al., 2004) including other yeasts used as biological control agents, which would retain their effectiveness in controlling fungal diseases (Abadias et al., 2005).

Thus, the method for obtaining an effective composition comprising Rhodotorula minuta for biological control of C. gloeosporioides consists of the following stages: the production of adequate quantities of biomass, optionally the recovery and resuspension of the cell pack, optionally the formulation by the addition of a support, and drying of these formulations.

First stage: production of biomass, the production process of R. minuta starts by developing the inocula in Petri dishes, placing them in 2.8 L stirred flasks, then transferring the content to a seeding bioreactor to obtain seed culture. The pH is adjusted to an initial value between 4 to 9, then it is stirred at between 100 to 300 rpm, to obtain good mass and heat transfer; with control of the proper temperature for growth, for example 20 to 30° C., with sufficient aeration in the first 4 hours, preferably 0.5 Volumes of Air by Volume of Medium per Minute (VMM) and increasing this value between 50 and 100% of the initial value for the following 16 hours of culture. After this time the seed culture is transferred to the production bioreactor containing mineral medium enriched with yeast extract, with an initial pH between 4 and 9. It is stirred at between 100 to 400 rpm to obtain good mass and heat transfer; with control of the proper temperature for growth, for example, 20 to 30° C., with sufficient aeration to ensure adequate growth, preferably 1 VVM. Culture time may vary depending on the parameters used for culture, but can range from 40 to 65 h. This time is required for R. minuta yeast to be in stationary phase (see example 1), because several authors (Werner-Washburn et al., 1993, Panek and Panek, 1990; Plesset theory et al., 1987) reported that another type of yeast, i.e. Saccharomyces cerevisiae, are more resistant to various kinds of stress stationary phase. After this time, the number of viable cells of R. Minuta obtained is on the order of 1×10⁹ CFU/mL.

Second stage (optional): Recovery and resuspension of the cell pack. Consists of retrieving the cell pack of the Rhodotorula minuta yeast to remove the largest quantity of water from the culture medium. This can be done by using individual centrifugation or microfiltration operations (Bhosale et al., 2003). For centrifugation, for example, a Sharples type tubular centrifuge can be used. In this case it will be necessary to resuspend the cell pack in a neutral pH buffer such as phosphate, to obtain a concentrated suspension of Rhodotorula minuta yeast.

Third stage (optional): formulation. Consists of the formulation of R. minuta biological control agent by adding a protective or carrier support, which can utilize various supports or carriers including those that are based on a flour with high starch content, such as maltodextrin, corn flour or corn starch (see Example 7).

In this invention, a solid dry composition of R. minuta cells can be obtained by adjusting the suspension to create a 3 to 20% concentration of solids (see example 2). This suspension serves to feed a dryer.

Fourth stage: drying. This stage can be performed, for example, in a dryer by spraying, with an input temperature between 100 to 160° C. and an output temperature between 50 and 70° C. Biomass concentrations of 1% to 15% can be used, preferably 10%; with input temperatures between 40 and 180° C. and an output temperatures range between 33 and 82° C.

For biological control agents, the application of the drying processes is not obvious; as cited in the antecedents, it was not convenient to apply various drying processes, including one with minor thermal aggression such as lyophilization, for achieving high yield recovery of viable cells, which in turn are effective against pathogens (Abadias et al., 2001, Jones et al., 2004; Abadias et al., 2005). Therefore, the processes covered by this invention are novelty.

Drying by lyophilization is very costly and, as mentioned above, has not been successful when applied to yeasts used as biological control agents. Fluidized bed drying, which has been widely studied and used to produce yeast for baking, requires lower equipment costs than that needed for lyophilization, but is more expensive than that used for spray drying, in addition to requiring greater retention times and greater care in the relative humidity of the drying air. Finally, Silva et al. (2002) reported that spray-drying can be used to dry large quantities of biomass at a relatively low cost. Spray-dried powders can be transported cheaply and stably stored for extended periods of time. Moreover, Janisiewicz and Jeffers (1997) suggest that the greatest obstacle in the commercialization of biological control agents is the development of products formulated to have a stable shelf life, maintaining a similar effectiveness to that of fresh cells of the same agent. Consequently, applying spray-drying gives the possibility of creating large quantities of products made with biological control agents with long shelf life and effectiveness in commercial orchard application in the field.

The spray-drying of R. minuta harvested in stationary phase yielded solid compositions with more conveniently long shelf life than the previously developed liquid formulations (Patiño-Vera et al., 2005) (see example 3), with a shelf life under refrigeration of up to a year for optimum subsequent use as biological control agent of C. gloeosporioides, causal agent of mango anthracnose. This extended shelf life may be due to the fact that by concentrating and drying the cells, the activity of water (a_{w}) is significantly reduced and certain substances toxic to by R. minuta, such as hydrogen peroxide and formaldehyde (compounds derived from the metabolic activity of yeast) are decomposed or evaporated. On the other hand, the solid and dry compositions of R. minuta obtained by the method of this invention were not affected in their ability to biologically, control mango anthracnose, unlike what occurred in other yeast drying cases reported in the antecedents.

Using the method of this invention it is possible to produce a solid, dry composition, effective in the biological control of C. gloeosporioides with a refrigerated shelf life of at least one year, characterized in that it includes viable R. minuta cells.

Moisture levels of dry yeast below 5% (w/w) are not recommended, because the cells can suffer irreversible biochemical damage (Masters, 1985) and, with the drying procedure proposed in this invention, solid dry compositions, comprised of R. minuta cells can be obtained with a minimum moisture of 5.23±0.59% and a maximum of 7.68±0.59% (see example 2).

Practical application of biological control agents usually employs doses with high concentrations of viable cells (about 10⁷ to 10⁸ CFU/mL) (Janisiewicz and Korsten, 2002); therefore commercial presentations of compositions with biological control agents must have at least one order of magnitude more in viable cell concentrations, to carry the least amount of solvent possible and thus minimize the transportation, storage and handling costs of the composition. This invention has developed a solid, dry, effective composition, with a viable cell concentration of at least 1×10⁹ CFU/g of R. minuta yeast.

The minimum shelf life recommended for the compositions with biological control agents is 6 months (Janisiewicz and Korsten, 2002), to be consistent with routine handling and storage practices in the agrochemical market. The solid dry composition, effective in the biological control of C. gloeosporioides, has a shelf life under refrigeration of at least one year, and is characterized as comprising viable cells of R. minuta.

As mentioned above, several authors have reported the inconsistency in the effectiveness of biological control agents when applied in commercial orchards (Janisiewicz and Jeffers, 1997; Janisiewicz and Korsten, 2002; Guetsky et al., 2001) and the benefits that can be achieved using mixtures of biological control agents (Janiciewicz and Jeffers, 1997; Janisiewicz and Korsten, 2002; Spadaro and Gullino, 2004). Therefore, this invention also includes a composition characterized in that it comprises a second biological control agent, and in that it requires lower doses of each biological control agent which, in independent application, can obtain similar levels of biological control. As reported by the authors of this invention (Carrillo-Fasi et al., 2005) Bacillus subtilis was the biological control agent with suitable characteristics for mixing with R. minuta, consequently, the dry solid composition of this invention, which includes R. minuta, can be mixed with dry solid compositions of B. subtilis, either pre-mixed or presented in separate packaging with the mixture and resuspension being made before application.

This type of dry solid composition of R. minuta produced in semi-commercial quantities was applied in mango orchards of the State of Sinaloa, Mexico, in a mixture with Bacillus subtilis (effective biological control agent against many fungal diseases). To obtain the necessary quantities of Bacillus subtilis, spores of this microorganism were produced as reported by Carrillo-Fasi et al. (2005).

R. minuta compositions showed better control of the severity of anthracnose in comparison with the commercial chemical fungicide Benomyl (commercial name: Benlate), as well as in liquid compositions with R. minuta as the only biological control agent, as well as those also containing B. subtilis in liquid or solid compositions. The applications of R. minuta+B. subtilis compositions, liquid or solid, used in commercial mango orchards cv. Kent, managed to reduce the severity of anthracnose up to 86.7%, compared with fruit in a control treatment (in which only irrigation water was applied) after 15 days of storage (see example 4 and 6). An additional advantage of the use of the mixture of antagonists is that it reduces, by up to two orders of magnitude, the viable microorganisms needed, compared to cases in which antagonists were used separately. The use of biological control agents offers the possibility to export agricultural products to countries within the European Economic Community and others like Japan, where the value of the fruit is higher than that quoted in the United States. This is a substantial improvement to the state of the art because there were no reports where R. minuta yeast was used in solid composition or mixed with liquid or solid formulations of B. subtilis for the control of fungal diseases of plants of commercial interest, particularly in fruit trees.

Thus, the claim is for a method for biological control of Colletotrichum gloeosporioides which includes at least one pre-harvest application of effective doses of a solid, dry, effective composition, including the biological control agent Rhodotorula minuta.

Ideally, the method for biological control of C. gloeosporioides covered by this invention implies that the composition is applied by spraying the entire aerial part of the plant to be treated.

According to this invention, solid, dry compositions of R. minuta, either used alone with this yeast or applied in mixture with Bacillus subtilis, are very useful in the field due to their effectiveness in control of mango anthracnose caused by C. gloeosporioides, without impairing the quality of the fruit, because they do not affect other fruit quality indices such as acidity, pH and total soluble solids (see example 5), while also improving its shelf life by losing less water during post-harvest storage, making them suitable. The results of the method for the biological control of C gloeosporioides are as good or superior to those obtained by chemical treatment (see example 4 and 6), which makes them suitable for use in the agrochemical market.

C. gloeosporioides is a causal agent of anthracnose in tropical and subtropical crops such as: mango fruit (Mangifera indica), papaya (Carica papaya L.), avocado (Persea americana), soursop (Annona muricata), mandarin (Citrus reticulata, C. unshiu and C. reshni) and rough lemon (Citrus jambhiri Lush) (Arauz, 2000; Gamagae et al., 2004, Freeman et al., 1998, Alvarez et al., 2004; Contreras and Rondon, 1985); smaller fruit such as strawberries (Fragaria ananassa L.) (Freeman et al., 1998); plants whose interest is their flowers, such as orchids, (Orchidaceae); even tuber crops like the yam (Dioscorea sp.) are affected (Pérez-Castro et al., 2003); among other things, this invention uses the case of the mango to demonstrate the effectiveness in the field of solid dry compositions including R. minuta, without implying that it only works with mango as target crop. These compositions are effective in the biological control of C. gloeosporioides and are therefore useful in the biological control of diseases caused by C. gloeosporioides in mango or any other crop.

### Materials and Methods

Strain of the pathogen Colletotrichum gloeosporioides, the phytopathogen fungus was isolated from mango fruit with anthracnose and identified according to its morphological characteristics (Freeman et al., 1998). The strain is maintained in potato dextrose agar medium (PDA) (BD Bioxon, Mexico) and stored at 4° C.

Strain of the yeast biological control agent Rhodotorula minuta: this yeast forms spherical colonies, bulging and bright pink in color, with smooth edges and unable to form spores or mycelium on solid medium NYDA (nutrient broth 8 g/L, yeast extract 5 g/L, dextrose 10 g/L and agar 18 g/L). It belongs to the Deuteromicetes; Order Criptococcales, Family Criptococcaceae; subfamily Rhodotoruloidea, genus Rhodotorula (Girard and Rougieux, 1964). The strain of Rhodotorula minuta was kept under refrigeration at 4° C. in inclined tubes with potato-dextrose-agar medium (PDA) (BD Bioxon, Mexico). Since in this invention mango is used to illustrate application in the field, we proceeded to isolate the yeast in the phyllosphere of the target plant (since it is common in the area of biological control), following the procedure reported by the authors of this invention in Patiño-Vera et al. (2005). Similarly, one can isolate strains of R. minuta in the phyllosphere from other crops for which C. gloeosporioides is pathogenic. However, there also exist available strains in the ATCC, with 13 entries in their Fungi, Yeasts & Genetic Stock catalog, for example, strains numbers 10658, 14926, 16731.16732, 16733, 16741, 208876, 2776, 32769 and 36236.

Culture mediums for R. minuta. Inocula and seed mediums (PYD) (g/L): potato starch 4.0, dextrose 20.0 and yeast extract 5.0; production medium (mineral-enriched medium) (g/L): 34.4 dextrose, yeast extract 5.0, 7.4 of (NH₄)₂HPO₄>2787 of KH₂PO₄, 2.05 of MgSO₄.7H₂O, 0.1 of NaCl, 0.009 of FeSO₄.7H₂O, 0.055 of CaCl₂, 0.01 of CuSO₄.5H₂O, and 0.0076 of MnSO₄.H₂O.

Strain of the biological control agent Bacillus subtilis. The strain of Bacillus subtilis is characterized by rhizoid morphology colonies, wavy edges, grainy surface and mucoid consistency (becoming dry and brittle when old), when grown on a solid medium. The color of the colonies is bright white-cream (in young colonies) and opaque cream (when aging). Staining with Gram differential tincture is positive, with formation of flagellate bacilli. To reactivate the strain we reseed a tube of glycerol in a Petri dish with sterile medium YPG (yeast extract 10 g/L, peptone 10 g/L and dextrose 10 g/L, with 15 g/L agar), then incubate at 30° C. for 24 hours. Since this invention uses mango to illustrate the application in the field, we proceeded to isolate the bacteria from the phyllosphere of the target plant (since it is common in the area of biological control), following the procedure reported by the authors of this invention in Fasi-Carrillo et al. (2005). Similarly, one can isolate strains of B. subtilis in the phyllosphere of other crops for which C. gloeosporioides is pathogenic. However, there also exist available strains in the ATCC, with more than 200 entries including numbers 31578, 33677, 35148, 39085, 39086, 39087, etc.

Culture mediums for B. subtilis. Inocula and seed mediums (YPG) (g/L): yeast extract 10, peptone 10 and dextrose 10; production mediums (g/L): 4.00 in (NH₄)₂SO₄, 5.32 of K₂HPO₄, 6.40 of KH₂HPO₄, 0.40 of MgSO₄.7H₂O, 0.005 of MnCl₂, 0.040 of CaCl₂, 0.030 of FeSO₄.7H₂O and dextrose 10.0.

Phosphate buffer solution (for liquid formulation) (g/L): sodium chloride 8.0, monobasic potassium phosphate 2.0, potassium chloride 2.0 and dibasic sodium phosphate 2.9.

Viable cell count. It was made manually by the plate count method, quantifying the Colony-Forming Units (CFU).

Determination of reducing sugars. To determine glucose concentration, 1 mL was taken from the fermentation broth, which was centrifuged for 3 minutes at 13,000 rpm in an Eppendorf centrifuge; dextrose was the determined from the supernatant, with Yellow Spring Scientific Instruments (YSI) enzymatic analyzer, model 2700. This analyzer uses an enzyme (glucose oxidase) which carries out a catalytic reaction that produces hydrogen peroxide which, when oxidized on a platinum anode, produces an electric Signal, which is directly proportional to the concentration of dissolved glucose in the sample.

Determining the shelf life of liquid formulations. After obtaining the liquids, they are stored under refrigeration and tested periodically by taking 1 mL of liquid formula and applying the procedure for viable cell count (CFU) over the desired monitoring period.

Determining the shelf life of solid formulations. After the various yeast suspensions are dried, they are stored in a cold room at 4° C. and viability is assessed each month. The procedure consists of weighing 100 mg of powder, resuspending it in a test tube with 10 mL of sterile saline solution (using NaCl) 0.85%, perfectly homogenizing it (to re-hydrate the cells) and making the corresponding dilutions to determine the CFU/g by the same process of viable cell count.

Determination of moisture content of solids formulations. Weigh in triplicate 100 mg of solid formulation on previously tared aluminum trays; later, place in an oven to dry at 105° for 2 h and then store in a dessicator to cool and obtain the constant weight. The moisture is obtained by weight difference before and after drying.

Determination of water activity, water activity of the samples was determined using an electric hygrometer, “Novasina AW Sprint” brand, model TH500, according to the manufacturer's instructions.

Application of treatments in the field. To verify field effectiveness we used an eight-year old commercial mango orchard cv. Kent, located in El Rosario, Sinaloa, Mexico. We selected the trees homogeneously to distribute them for each treatment (three trees per treatment). In each treatment there was a monthly application (five in total), using a backpack pump sprinkler operated at 400 pounds of pressure. We started during flowering (February) until the harvest (June or July), spraying suspensions of fresh or dried cells (previously hydrated), produced in semi-industrial fermenters. We sprayed 7 L of cell suspension on each selected mango tree, until all the foliage was wet. As a control treatment, we sprayed 7 L of irrigation water.

Quantifying the severity of anthracnose. For each treatment, 100 mango fruits were harvested at physiological maturity (Báez et al., 1993), and were stored under simulated marketing conditions at 20° C. and 85% relative humidity. We evaluated the severity of the disease after a minimum of 15 days of storage, using the presence of symptoms as indicator of the disease (anthracnose) in the fruit. For this purpose, we used an adaptation of the hedonic scale proposed by Smoot and Segall (1963), where: 0=healthy, 1=traces (chlorotic spots), 2=light (dark lesions 1-5 mm in diameter), 3=medium (dark lesions larger than 6 mm in diameter), and 4=severe (dark sunken lesions and presence of fungal structures).

Assessing the quality of the fruit. Weight loss was assessed every third day by difference in weight with respect to the initial weight of the fruit, expressed as a percentage (Diaz-Pérez, 1998). The pH, titratable acidity (TA) and total soluble solids (TSS) were quantified according to the methodology proposed by the AOAC (1998) at 0, 6 and 12 days of storage. To determine the pH and acidity we used a Mettler automatic titrator (model DL-21) and the results were expressed in units of pH and percentage of citric acid, respectively. Total soluble solids were determined with a Leica Abbe Mark II type refractometer, temperature-compensated and previously calibrated with pure water. The results were expressed in Brix degrees (°Brix). For quality parameters, a fruit was selected as an experimental unit and five replications were used.

Statistical analysis. The study of the severity of the disease in mango fruits was analyzed using a completely randomized block design. Data from the evaluations of each treatment were subjected to unilateral variance analysis by Friedman ranges (p<0.05) using the SAS statistical system (SAS, 1998). The difference between treatments was determined by the Tukey test (p=0.05). For differences in the analysis of variance (ANOVA) of quality variables, the mediums were separated by Tukey multiple comparison test with a probability of error of 5%, using the Minitab statistical program version 13.1.

### EXAMPLES

The following examples better illustrate this invention, but of course without restricting its scope.

### Example 1

### Production of Cells of R. minuta in Stationary Phase in 10 L Stirred Fermenter Operation

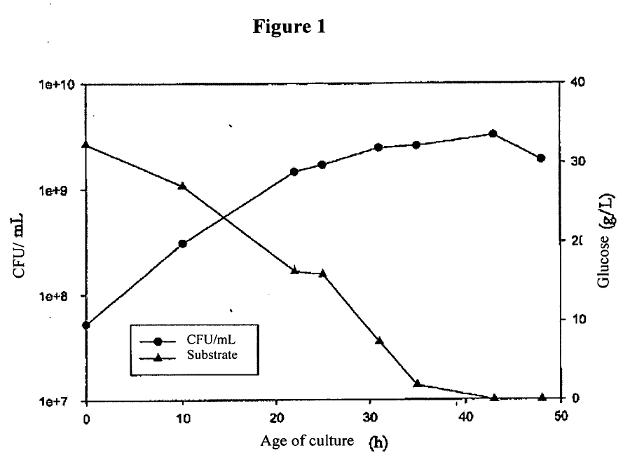

We started with the spread of the Rhodotorula minuta strain in Petri dishes with PDA medium, transferring the yeast from a previously inoculated and incubated inclined tube with abundant growth of yeast, using a microbial seed loop, following the usual microbiological techniques to ensure sterility and avoid contamination of materials and culture mediums. The Petri dishes were sown by streak and incubated at 29° C.±1 for 24 for 48 hours. When there was a noticeable growth of yeasts throughout the streak, three roasts were transferred to a 250-mL Erlenmeyer flask with PYD mediums and incubated stirring at 200 rpm at 29±1° C. for 24 hours. At least two flasks of 250 mL were prepared to transfer 50 mL of this pre-inoculum to a 2.8 L flask or wide-bottomed Fernbach, with 450 mL of sterile PYD mediums, and then incubated stirring at 200 rpm at 29±1° C. for 24 h. At least two Fernbach flasks were prepared to transfer one liter of the inoculum to a 14 L fermenting jar, with 10 L of Mineral Enriched Medium (MEM). The jar was equipped with three Rushton turbines with 6 flat blades and a diffuser section. It was stirred at 240 rpm at 29±1° C. with aeration of 10 L/min for 48 hours. To know the time of culture in which the yeast R. minuta is found in stationary phase, we determined its growth kinetics and consumption of glucose (main source of carbon from the production medium). As shown in FIG. 1, it is possible to recognize when this yeast is in stationary phase by measuring the consumption of glucose. When glucose is in a concentration range between 0 and 2 g/L (between 35 and 48 h of culture), it is clear that the growth of R. minuta has been arrested and practically kept at the same concentration of CFU/mL; consequently, it is stationary phase, in which it is possible to successfully dry this yeast.

### Example 2

### Formulation and Spray Drying of Rhodotorula minuta

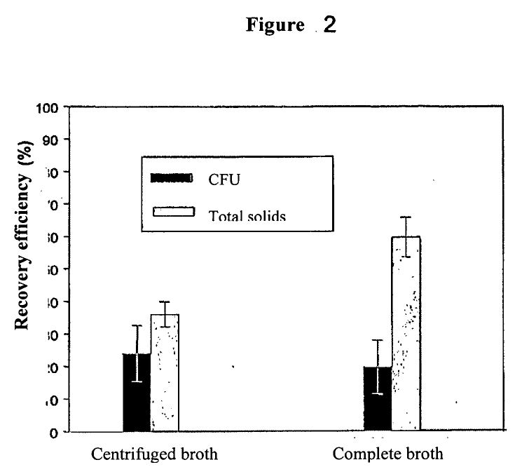

Two different methods were used to develop the formulation and drying process of a solid composition effective in the biological control of mango anthracnose with Rhodotorula minuta. The first condition consisted of obtaining cell paste by centrifuging the broth harvested at the end of the fermentation process (as described in example 1) using a MiniSharples tubular centrifuge (CL-1-1 Model), with a bowl diameter of 1.75 inches in an operating range of 8000 to 12,000 rpm. Subsequently, the paste was resuspended in phosphate buffer, so that the suspension had a concentration of 6% total solids. In the second condition, the dryer was supplied with complete culture broth, as harvested from the fermenter, which contains a total solid concentration of 3%. All drying experiments were conducted under the same operating conditions: 120° C. input temperature, 50-52% relative humidity (RH), 180 mL/min feeding flow of the liquid suspension, 50-60° C. output temperature, total solid concentration of approximately 3 or 6% from R. minuta biomass harvested in stationary phase (see example 1). The results for this example are shown in Table 1 and FIG. 2.

TABLE 1
Summary results of the drying of solid formulations

| Parameter/condition | 1) Centrifugation | 2) Complete |
|---|---|---|
| Input temperature (±2° C.) | 120 | 120 |
| Flow (±3 mL/min) | 180 | 180 |
| Output temperature (±5° C.) | 55 | 55 |
| % of total solids in the feed | 5.61 ± 2.64 | 3.35 ± 0.61 |
| Dry product obtained (g) | 25.6 ± 2.36 | 42.8 ± 4.38 |
| Total cells/g | 8.44 × 10¹⁰ | 6.92 × 10¹⁰ |
| Standard deviation (%) | 3.91 | 14.49 |
| UFC/g of dry product | 3.08 × 10¹⁰ | 1.32 × 10¹⁰ |
| Standard deviation (%) | 31.26 | .20.01 |
| UFC recovery efficiency (%) | 23.9 ± 8.6 | 19.6 ± 8.27 |
| Solid recovery efficiency (%) | 35.9 ± 3.78 | 59.4 ± 6.14 |
| Water activity | 0.337 ± 0.033 | 0.317 ± 0.014 |
| Moisture (%) | 5.23 ± 0.59 | 7.68 ± 0.68 |

As shown in Table 1 and FIG. 2, the process of spray drying affected the proportion of viable R. minuta cells recovered; an average of 23.9±8.6% viable cells were recovered in the centrifugation of the broth, while 19.6±8.3% of them were recovered from the complete broth. Regarding the efficiency of the recovery of solids, the lowest values were presented in the case where the culture broth is centrifuged, because it contained fewer dissolved solids (as mineral salts), which were eliminated in the prior centrifugation process.

On the other hand, the efficiency of viable cell recovery (CFU) is slightly lower when the broth is fed completely (without centrifugation), than when fed the resuspended cell pack. However, the concentration of viable cells per gram (measured as CFU/g) is still on the order of 10¹⁰ and the efficiency of recovery of solids is high. Therefore, there are two variations of the process for obtaining solid formulations of R. minuta. The first is feeding the spray dryer with the complete harvested broth (without using centrifugation), which requires less investment in equipment and simplifies the process, lowering operating costs. The second process is accomplished by centrifugation and resuspending the R. minuta cell paste in phosphate buffer, which achieves a greater number of live cells per gram (2.3 times greater than without centrifugation) in the dry product recovered from the dryer. This would allow using a significantly smaller amount of product in order to apply to the same number trees. The latter composition is characterized in that, when centrifuging and discarding the supernatant, much of the residual soluble solids of the R. minuta culture broth are removed

As indicated by the data in Table 1, the water activity (a_{w}) of the solid compositions obtained by the method of this invention is relatively low. This means that the yeast cells are left with very low metabolic activity, since most microorganisms are unable to thrive in environments with very low a_{w}, so they die or become dehydrated, or become dormant for indefinite periods of time (Madigan et al., 1999, Paul et al., 1993). On the other hand, the moisture content of solid formulations is within the appropriate range, since it is reported that moisture content between 5-8% does not cause irreversible damage to the metabolic functions of microbial cells (Masters, 1985).

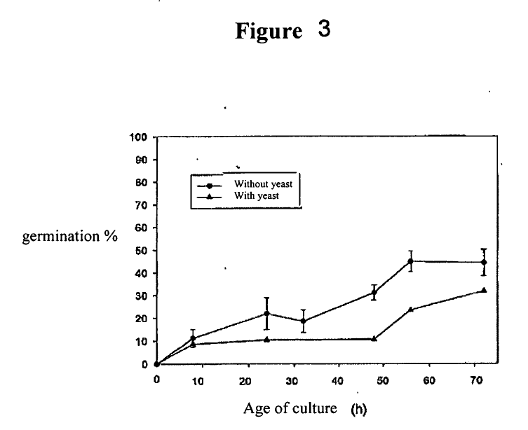

To verify that the solid and dry composition with R. minuta, the subject of this invention, retains the capacity of biological control of the fungus Colletotrichum gloeosporioides, causal pathogen of anthracnose in mango, a bioassay was conducted “in vitro”, using microplates with 24 wells, similar to what was reported by Janisiewicz et al., (2000). This bioassay provided the curves of the germination percentage of the phytopathogen. As can be seen in FIG. 3, during 50 hours of culture, the number of conidia remained almost constant for both cases, giving about 30% of them time to germinate (in the control) and less than 10% in the bioassay with the solid dry composition with R. minuta.

In FIG. 3, comparing the control experiment (without the dry solid composition and R. minuta) and the one with yeast, one can clearly observe the effect of inhibiting the germination of the conidia of the phytopathogenic fungi C. gloeosporioides, produced by the dry solid composition of R. minuta. The difference between the two conditions was up to 25%, so it can be said that the dry solid composition obtained by the method of this invention, subject of this invention, retained virtually the same antagonist effect, in vitro, as a liquid composition with fresh cells of the same biological control agent.

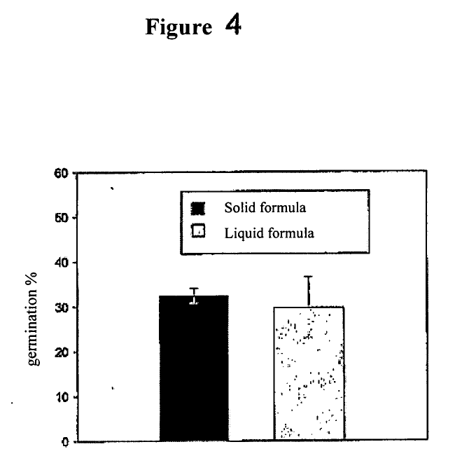

On the other hand, it was also confirmed that the yeast of the dry solid composition presented a degree of antagonism similar or equal to the fluid formulation, previously reported by our research group (Patiñio-Vera et al., 2005). The results of the germination percentage, at 56 hours of culture, are shown in FIG. 4. This figure shows that the solid dry composition had an almost equal inhibition effect on the germination percentage (no significant difference) as the liquid composition, with the same dose of R. minuta of 10⁸ UFC/mL.

### Example 3

### Shelf Life of the Solid Formulation Developed, Containing R. minuta, in Comparison with the Liquid Formulations

To compare the shelf life of the two types of formulations, a follow-up of the concentration of viable Rhodotorula minuta cells was conducted, in both the liquid and solid compositions developed in this invention.

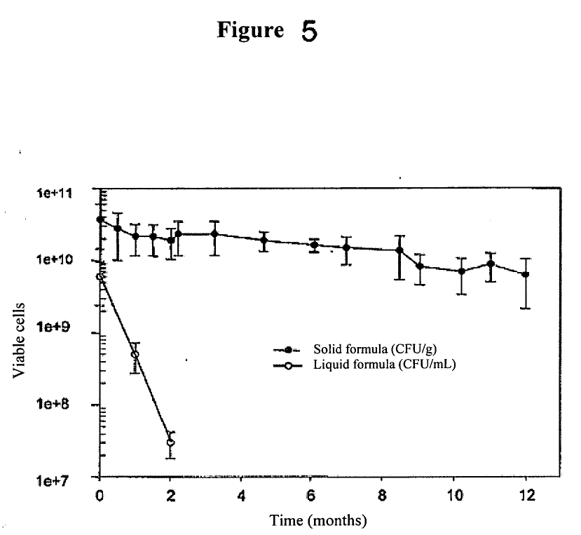

Fresh liquid compositions were obtained as reported by Patiño-Vera et al. (2005). After fermentation, the cell pack (obtained by centrifugation in Minisharples equipment) was resuspended in phosphate buffer with a ratio of 0.333 mL/g moist paste. The shelf life of the liquid and solid formulations was obtained by storing at 4° C. and, as shown in FIG. 5, the concentration of viable cells in the solid composition, subject of this invention, was maintained at levels of 10¹⁰ CFU/g for a significantly longer period of time (10 months or more), than in the liquid composition (in phosphate buffer). The liquid formulations lose almost 90% of their concentration of viable cells in the first month, while the solid formulation lost only 5% over the same period. In the second month, the concentration of Rhodotorula minuta viable cells in liquid formulations decreases over two orders of magnitude, while the solid dry composition still has 80% of the initial viable population.

This could be due to the fact that the drying process removes a large part of the water content and volatile toxic substances with low molecular weight, in addition to presenting diffusional barriers (by gel formation) and limiting cell metabolism, because it is reported that the dehydration caused by drying reduces the availability of water within the cells, so they enter a latent state, in which metabolism is almost completely stopped (Paul et al., 1993).

The solid dry compositions developed and described in this invention maintained a high concentration of viable cells (10¹⁰ CFU/g) for up to 12 months of storage (FIG. 5). This time is sufficient and acceptable for a biological control product to be marketed (Pusey, 1994).

### Example 4

### Field Tests with Liquid Compositions of R. minuta as the Sole Biological Control Agent and with a Second Biological Control Agent, B. subtilis, which Illustrates the Effectiveness of these Microorganisms as Biological Control Agents Against Colletotrichum gloeosporioides.

Field applications were made in pre-harvest, in accordance with the procedures described in Materials and Methods. The doses and formulations used are presented in table 2.

TABLE 2
Treatments and doses applied in a mango orchard (Mangifera indica)
cv. Kent to control anthracnose (caused by C. gloeosporioides).

| | | |
|---|---|---|
| Treatment | Description | Dose |

| | | | |
|---|---|---|---|
| 1 | Rhodotorula minuta | 10⁸ | UFCmL⁻¹ |
| 2 | Bacillus subtilis | 10⁶ | UFCmL⁻¹ |
| 3 | R. minuta + B. subtilis | 10⁶ + 10⁴ | UFCmL⁻¹ |
| 4 | Benomyl | 0.5 | g L⁻¹ |

| | | |
|---|---|---|
| 5 | Absolute control | — |

The data on the severity of cumulative anthracnose (expressed in % of severity), after 15 days of storage of the fruit is presented in table 3. Values of up to 60.5 of severity of the disease for the fruits of the absolute control and 41.7 for the chemical treatment were obtained. The results indicated (see table 3) that with the combined application of antagonists, greater control of anthracnose is obtained (lower severity of illness, to just 8.0 in terms of severity), finding significant differences between treatments and showing drastically lower severity values with respect to the application of chemical compound and the absolute control. An important advantage of using the R. minuta composition as the first biological control agent and B. subtilis as a second, subject of this invention, was that it allowed achieving higher levels of anthracnose control using concentrations two orders of magnitude smaller than in the case of separate application of antagonistic microorganisms (10⁸ to 10⁶ CFU/mL in the case of yeast and 10⁶ to 10⁴ CFU/mL for bacteria, see table 2); this makes this treatment more attractive for commercial application since it would be necessary to apply 100 times less cells of each of these biological control agents and still achieve a better control of anthracnose. The reduction in anthracnose severity in combined antagonist treatment indicates a possible synergistic effect between the yeast and bacteria. The application of antagonistic microorganism mixtures has reduced variability and improved the efficiency of biological control in many systems that include fruit pathogens (Guetsky et al., 2001). However, this is the first time that control of anthracnose caused by C. gloeosporioides was reported, proven at a semi-commercial level, using mango as target culture. In this example, the compositions used in the Kent mango were clearly more effective than chemical treatment, possibly because the antagonists colonized the surface of leaves and fruits and thereby minimized early or latent infections of mango fruit.

TABLE 3
Range of anthracnose severity on mango fruit (Mangifera indica)
cv. Kent pre-processed absolute control.

| | | % of severity after 15 |
|---|---|---|
| Treatment | Description | days of storage |
| 5 | Absolute control | 60.5 *a |
| 4 | Benomyl | 41.7 b |
| 1 | Rhodotorula minuta | .7 c |
| | (10⁸ UFC/mL) | |
| 2 | Bacillus subtilis | 12.4 d |
| | (10⁶ UFC/mL) | |
| 3 | R. minuta + B. subtilis | 8.0 e |
| | (10⁶ UFC/mL + 10⁴ UFC/mL) | |

Values with different font have a statistically significant difference (Tukey, p < 0.05).

### Example 5

### Effect of Application of the Compositions of Biological Control Agents Developed on the Quality of the Fruit Produced

The same compositions presented in table 2 were used to study the effect of the application of biological control agents, subject of this invention, on the quality of mango fruit cv. Kent.

One of the most important parameters for evaluating the quality of mango fruit is weight loss during storage. In analyzing the behavior of this variable it was observed (see table 4) that, at the beginning of the marketing stage (day 3 of storage), all fruits showed a similar pattern with a weight loss of less than 1%. It is from the ninth day of storage at 20° C. that statistical differences in weight loss compared with the control were observed (p<0.05). In the fifteenth day values were 4.6 and 4.3% fruit weight loss for the control and chemical treatment, respectively, and less than 3.6% for the fruits treated with the composition that includes yeast (R. minuta 10⁸ CFU/mL).

TABLE 4
Changes in weight loss of mango fruit (Mangifera indica) cv. Kent, during
storage for up to 15 days at 20° C. and 85% relative humidity, treated
with biological antagonists, chemical fungicides and absolute control.

| | Weight loss (%) | | | | |
|---|---|---|---|---|---|
| | Day | | | | |
| Treatment | 3 | 6 | 9 | 12 | 15 |
| Rhodotorula minuta | 0.8 a* | 1.5 a | 2.2 b | 3.0 b | 3.6 b |
| 10⁸ UFC mL⁻¹ | | | | | |
| Bacillus subtillis | 0.8 a | 1.6 a | 2.5 ab | 3.3 ab | 4.1 ab |
| 10⁸ UFC mL⁻¹ | | | | | |
| R. minuta 10⁶ + B. subtillis | 0.7 a | 1.5 a | 2.2 b | 3.1 b | 4.0 ab |
| 10⁴ (UFC mL⁻¹) | | | | | |
| Benomyl | 0.9 a | 1.6 a | 2.4 ab | 3.4 ab | 4.3 a |
| Absolute Control | 0.9 a | 1.6 a | 2.6 a | 3.7 a | 4.6 a |

*Values with the same font between columns are statistically equal. (Tukey, p < 0.05).

Other quality parameters evaluated were the pH, acidity and total soluble solids content in the fruit. In analyzing these it was shown that treatment with both compositions (R. minuta and R. minuta+B. subtilis) had the lowest pH values, being statistically different from the fruits tested at the beginning of storage (table 5). On the other hand, there was no relation between pH and the results of the content of citric acid. The pH tended to rise during the ripening of the fruits, associated with a decline in titratable acidity. At 12 days of storage, treatment of the mixture of B. subtilis and R. minuta had the highest pH value. The percentage of citric acid began with high (and statistically the same) values and was significantly reduced after 12 days of storage with the B. subtilis treatment and the R. minuta+B. subtilis mixture, which had acidity values of 0.06 and 0.04, respectively, where the absolute control presented the highest value (0.14). The greatest reduction in the acidity of fruit treated with biological antagonists is probably due to fact that the microorganisms use organic acids for respiratory activity (Guadarrama and Ruiz, 1992). Similar to pH, total soluble solids content was not affected by the application of different treatments (table 5), which presented, on average, an increase of 8 to 14°Brix during the first twelve days of storage. According to Seymour et al., (1990), the increase in total soluble solids is due to the degradation of macromolecules to simpler sugar molecules, reporting for the Kent mango, a change of soluble solids from 7.7 to 16.1° Brix during ripening for 12 days at 20° C.

Consequently, applying the compositions of R. minuta and B. subtilis did not negatively affect the main fruit quality parameters of the Kent mango. On the contrary, it had a smaller weight loss during storage and, since this product is sold by unit weight (tons), producers would obtain higher gains from its marketing.

TABLE 5
Changes in pH, titratable acidity and total soluble solids of mango
fruits (Mangifera indica) cv. Kent during storage for 12 days at 20° C. and 85%
relative humidity, treated with biological antagonists, chemical fungicide and
absolute control.

| | pH | | | TA (% of citric acid) | | | SST (°Brix) | | |
|---|---|---|---|---|---|---|---|---|---|
| Treatment | 0 | 6 | 12 | 0 | 6 | 12 | 0 | 6 | 12 |
| Rhodotonula minuta | 4.2 b* | 4.2 a | 4.8 b | 0.78 a | 1.09 a | 0.08 ab | 7.7 a | 10.1 a | 14.5 a |
| Bacillus subtillis | 4.3 ab | 4.2 a | 5.1 ab | 0.52 a | 0.92 bc | 0.06 b | 7.9 a | 10.9 a | 15.0 a |
| R. minuta + B. subtillis | 4.2 b | 4.3 a | 5.5 a | 0.86 a | 0.76 c | 0.04 b | 7.2 a | 9.7 a | 14.4 a |
| Benomyl | 4.3 ab | 4.2 a | 5.1 ab | 0.58 a | 1.07 ab | 0.12 ab | 8.2 a | 9.6 a | 14.3 a |
| Absolute control | 4.4 a | 4.3 a | 5.3 a | 0.57 a | 0.76 c | 0.14 a | 8.9 a | 10.4 a | 12.6 a |

*Values with the same font within each column are statistically equal (Tukey, p < 0.05).

### Example 6

### Field Tests with Solid Dry Compositions of R. minuta, as the First Biological Control Agent, and a Second Biological Control Agent, B. subtilis

Field applications were made pre-harvest following the procedures described in Materials and Methods. The doses and formulations used are presented in table 6.

TABLE 6
Treatments and doses applied in a mango orchard (Mangifera indica)
cv. Kent to control anthracnose (caused by C. gloeosporioides).

| | | |
|---|---|---|
| Treatment | Description | Dose |

| | | | |
|---|---|---|---|
| 1 | R. minuta + B. subtilis | 10⁶ + 10⁴ | UFCmL⁻¹ |
| 2 | Benomyl | 0.5 | g L⁻¹ |

| | | |
|---|---|---|
| 3 | Absolute control | — |

The data on the severity of cumulative anthracnose (percentage of severity), one day after storage of the fruit can be seen in table 7, which shows that there is statistical difference between treatments (P<0.05). The treatment (R. minuta 10⁶ cfu/mL⁻¹+B. subtilis 104 CFU mL⁻¹), showed greater effectiveness in controlling anthracnose in mango fruit than Benomyl and the absolute control. The respective percentage of severity for the biological treatment was 23.1%, followed by chemical treatment (36.2%) and the absolute control (46.8%). A major advantage of the use of a R. minuta composition as first biological control agent and B. subtilis as a second, subject of this invention, was that it allowed achieving the highest anthracnose control levels by using concentrations two orders of magnitude smaller than in the case of separate application of antagonistic microorganisms (10⁸ to 10⁶ CFU/mL in the case of yeast and 10⁶ to 10⁴ CFU/mL for bacteria, see example 4); this confirms that the treatment with mix is more attractive for commercial application, since it would require applying 100 times fewer cells to each of these biological control agents and still achieve better anthracnose control than chemical fungicide. As mentioned earlier in this example, the dry solid compositions covered by this invention, used in the Kent mango, were clearly more effective than the chemical treatment, possibly because the antagonists colonized the surface of leaves and fruits and thereby minimized early or latent infections of mango fruit.

TABLE 7
Range of anthracnose severity on mango fruit (Mangifera indica)
cv. Kent treated in pre-harvest.

| Treatment | Description | % severity |
|---|---|---|
| 3 | Absolute control | 46.8 c |
| 2 | Benomyl | 25.0 b |
| 1 | R. minuta + B. subtilis (10⁶ | 23.1 a |
| | UFC/mL + 10⁴ UFC/mL) | |

*Values with different fonts present a statistically significant difference (Tukey, p < 0.05).

### Example 7

### Effect of the Formulation Through the Incorporation of Supports or Thermoprotectors in the Production of the Solid, Dry Composition with R. minuta.

Through the previously described fermentation process, ten liters of Rhodotorula minuta broth culture were obtained. Of these, 20 mL samples were taken to determine the content of total solids (TS) in duplicate, obtaining an average of 3.4% in the newly harvested broth. Subsequently, we added the amount of support necessary to have a 10% concentration of total solids and aliquots were taken for spray drying, at 4385 mL. Cornstarch (corn s.) was employed as support in the following composition: ST of the R. minuta broth 3.4%+6.6% cornstarch solids.

After preparing the above suspension, it was subjected to a spray drying process, with the following drying conditions: input temperature 120° C., feed flow 106 mL min⁻¹, dryer output temperature in the range of 60-65° C. and relative humidity range from 50 to 52% of drying air.

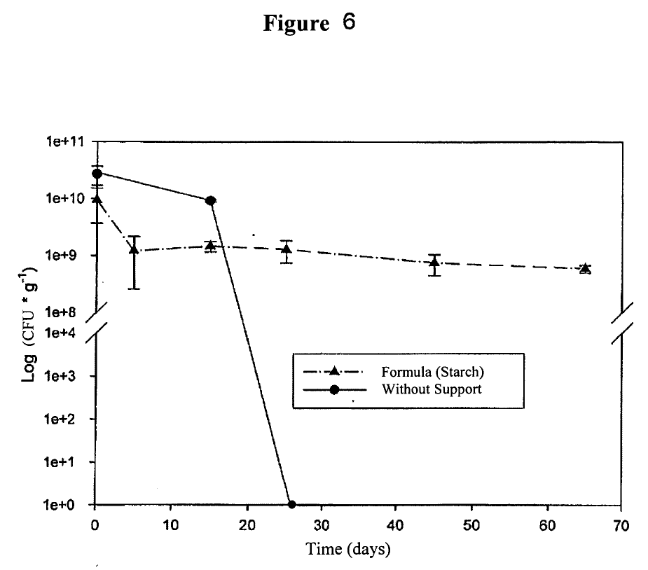

We determined the viability of the dry powder (9.43×10⁹ CFU/g), the percentage of solids recovered (87.5) and residual moisture (7.21%). To estimate the effect of the addition of a support on shelf life, some of the compositions of this invention were subjected to an accelerated aging process. For this purpose, we took a sample of 3 g of the dry formula in triplicate and placed it in an oven at 37° C., where it was maintained for a storage period of approximately 65 days. We periodically took samples of 0.1 g of the powder stored at different time intervals (at 5, 15, 25, 45 and 65 days), making a viable cell count (measured as CFU) per plate count. The data on the shelf life of the two formulations developed are shown in FIG. 6. The use of the support allowed maintaining a viable cell concentration (measured as CFU) of 1×10⁹ CFU*g⁻¹ at 37° C. for time much longer periods (65 days of storage), compared with formulations without the support, which after 24 days presented no more viable cells (measured as CFU).

As mentioned above, besides being the first to achieve dry solid compositions of R. minuta as biological control agents obtaining a high concentration of viable cells (measured as CFU) and high effectiveness in controlling fungal diseases, we are also the only ones to obtain a dry composition with a high concentration of viable cells (approximately 1×10⁹ CFU g⁻¹) that can be stored at a temperature of 37° C. for up to 65 days. This is very important because fruits like mango, papaya, avocado, strawberry, yam, etc. are produced in tropical or semitropical areas, with average temperatures near 37° C., allowing for better marketing of the compositions of this invention, since, if necessary, their exposure to 65 days at room temperature (37° C. on average) does not significantly affect their viability, thus presenting an additional technical and commercial advantage. As reported in the background, several authors have failed in their attempt to develop dry products (Wan-Yin and Mark, 1995, Jones et al., 2004), including the use of other yeasts as biological control agents, which retain their effectiveness in controlling fungal diseases, including storing at refrigeration temperatures (Abadias et al., 2005).

### REFERENCES

All publications, patents and applications listed here are incorporated by reference in their entirety in the description.
Abadias, M., Teixidó, N., Usall, J., Benabarre, A. and Viñas, I. (2001). Viability, efficacy, and storage stability of freeze-dried biological control agent Candida sake using different protective and rehydration mediums. Journal of Food Protection 64 (6): 856-861.
Abadias, M., Teixidó, N., Usall, J., Solana, C, and Viñas, I. (2005). Survival of the postharvest biological control yeast Candida sake CPA-1 after dehydratation by spray-drying. Biological control Science and Technology (8): 835-846.
Alvarez, E., Ospina, C. A., Mejia, J. F. and Llano, G. (2004). Characterization morfologica, patogenica y genetica del agente causal de la antracnosis (Colletotrichum gloeosporioides) en guanabana (Annona muricata) en el Valle de Cauca [Morphological characterization, pathogenicity and genetics of the anthracnose causal agent (Colletotrichum gloeosporioides) in soursop (Annona muricata) in the Valle del Cauca]. Fitopatologia Colombiana 28 (1): 1-8.
AOAC (1998) Official Methods of Analysis. 16th Edition. William S. (Ed). Association of Official Analytical Chemists. Chemists. Washington, D.C. CD ROM.
Arauz, L. F. (2000) Mango anthracnose: economic impact and current options for integrated management. Plant Disease 84 (6): 600-609.
Báez, S R, Siller, J., Bringas, E., and Baez, M. (1993). Determinacion de indices de madurez de los principales cultivares de mango producidos en Mexico. [Determination of ripening indices of the main cultures of mango produced in Mexico.] Proceedings of the Interamerican Society for Tropical Horticulture 37:148-154.
Benbow, J. M. and Sugar, D. (1999). Fruit surface colonization and biological control of postharvest diseases of pear by preharvest yeast applications. Plant Disease 83: 839-844.
Bhosale, P., Jogdand, V. V., and R. GADR V. (2003). Stability of 5 β-carotene in spray dried preparation of Rhodotorula glutinis mutant 32. Journal of Applied Microbiology 95: 584-590.
Cabrera, M. G., Galmarini, M. R., and Flachsland, E. (2002). Colletotrichum gloeosporioides patogeno de orquideas en el NE de Argentina [Colletotrichum gloeosporioides orchid pathogens in NE Argentina]. www.unne.edu.ar/cyt/2002/05_Agrarias/A-059.pdf
Carrillo-Fasio, J A, Garcia-Estrada, R S, Muy-Rangel, M A, Sañudo-Barajas, A., Marquez-Zequera, I., Allende-Molar, R., Patiño-Vera, M., Garza of -Ruiz, Z., and Galindo-Fentanes, E. (2005). Control biologico de antracnosis (Colletotrichum gloeosporioides Penz) y su efecto en la calidad poscosecha del mango en Sinaloa [Biological control of anthracnose (Colletotrichum gloeosporioides Penz) and its effect on the post-harvest quality of mango in Sinaloa.] Revista Mexicana de Fitopatologia 23 (1): 24-32.
Chand-Goyal, T. and Sports, R. A. (1998). Control of post-harvest fungal disease using saprophytic yeast. U.S. Pat. No. 5,711,946.
Contreras, V. N. and Rondon, A. (1985). Etiologia de la mancha parda de los citricos en Venezuela. [Etiology of brown spots of citrus fruits in Venezuela.] Agronomia Tropical 35 (1-3): 111-115.
Costa, E., Teixidó, N., Usall, J., Fons, E., Gimeno, V., Delgado, J., and Viñas, I. (2002). Survival of Pantoea agglomerans strain CPA-2 in spray-drying process. Journal of Food Protection 65 (1): 185-191.
De Jager, E. S., Hall, A. N., Wehner, F. C. and Korsten, L. (2001) Microbial ecology of the mango phylloplane. Microbial Ecology 42: 201-207.
Diaz-Pérez, J. C. (1998). Transpiration rates in eggplant fruit as affected by fruit and calyx size. Postharvest Biology and Technology 13: 45-49.
Freeman, S., Katan, T. and Shabi, E. (1998) Characterization of Colletotrichum species responsible for anthracnose diseases of various fruits. Plant Disease 82: 596-605.
Gamagae, S. U., Sivakumar, D., and Wijesundera, R. L. C. (2004). Evaluation of post-harvest application of sodium bicarbonate-incorporated wax formulation and Candida oleophila for the control of anthracnose of papaya. Crop Protection 23: 575-579.
Girard, H. and Rougieux, R. (1964). Tecnicas de Microbiologia Agricola [Agricultural Microbiology Techniques.] Editorial Acribia, Barcelona, Spain, p. 302.
Guetsky, R., Shtienberg, D., Elad, Y., and Dinoor, A. (2001). Combining biocontrol agents to reduce the variability of biological control. Phytopathology 91: 621-627.
Helbig, J. (2001) Field and laboratory investigations into the effectiveness of Rhodotorula glutinis (isolate 10391) against Botrytis cinerea Pers ex Fr. in strawberry. Journal of Plant Diseases and Protection 108 (4): 356-368.
Janisiewicz, W. J., and Jeffers, S. N. (1997). Efficacy of commercial formulation of two biofungicides for control of blue mold and gray mold of apples in cold storage. Crop Protection 16: 629-633.
Janisiewicz, W. J., and Korsten, L. (2002). Biological control of postharvest diseases of fruits. Annual Review of Phytopathology 40: 411-441.
Janisiewicz, W. J., Tworkoski, T. and Sharer, C. (2000). Characterizing the mechanism of biological control of postharvest diseases on fruits with a simple method to study competition for nutrients. Phytopathology 90: 1996-1200.
Jones, E. E., Weber, F. J., Oostra, J., Rinzema, A., Mead, A., and Whipps, J. M. (2004). Conidial quality of the biocontrol agent Coniothyrium minitans produced by solid-state culture in a packed-bed reactor. Enzyme and Microbial Technology 34: 196-207.
Koome, I. and Jeffries, P. (1993) Effects of antagonistic microorganisms on the post-harvest development of Colletotrichum gloeosporioides on mango. Plant Pathology 42: 230-237.
Korsten L, De Villiers, E. E., Wehner, F. C, and Kotzen, J. M. (1997). Field sprays of Bacillus subtilis and fungicides for control of preharvest fruit diseases of avocado in South Africa. Plant Disease 81 (5): 455-459.
Larena, I., Melgarejo, P. and De Cal, A. (2003). Drying of conidia of Penicillum oxalicum, a biological control agent against Fusarium Kilt of tomato. Journal of Phytopathology 161: 600-606.
P. Lodato, Huergo of Segovia, M., and Buera, M. P. (1999). Viability and thermal stability of a strain of Saccharomyces cerevisiae freeze-dried in different sugar and polymer matrices. Applied Microbiology and Biotechnology 52: 215-220.
Madigan, M. T., Martinki, J. M., Parker, J. (1999). Hongos [Fungi]. Eighth edition. In: Brock, Biologia de los Microorganismos [Biology of Microorganisms] Publisher Prentice-Hall, USA, pp. 170, 774-776.
Masters, K. (1985) Spray Drying Handbook, 4th. Ed, John Wiley, London.
Naidu, K. A. Venkateswaran, G., Vijayalakshmi, G. Manjula, K., Viswanatha, S., Murthy, K. N., Srinivas, L. and Joseph, R. (1999) Toxicological assessment of the yeast Rhodotorula gracilis in experimental animals. Zeitschriñ für Lebensmitteluntersuchung und-Forschung 208 (5-6): 444-448.
Panek, A. D. and Panek, A. C. (1990) Metabolism and thermotolerance function of trehalose in Saccharomyces: a current perspective. Journal of Biotechnology 14: 229-238.
Patiño-Vera; M., Jimenez, B., Balderas, K., Ortiz, M., Allende, R., Carrillo, A. and Galindo, E. (2005). Pilot-scale production and liquid formulation of Rhodotorula minuta, a potential biological control agent of mango anthracnose, Journal of Applied Microbiology 99:540-550.
Paul, E., Frages, J., Blanc, P., Goma, G. and Pareilleux, A. (1993). Survival of alginate-entrapped cells of Azospirillum lipoferum during dehydration and storage in relation to water properties. Applied Microbiology and Biotechnology 40: 34-39.
Perez-Castro, L, Baquero, M. J. and Beltrán-Herrera, J. D. (2003). Caracterizacion morfologica y patogenica de Colletotrichum sp. como agente causal de la antracnosis en name Dioscorea sp. [Morphological and pathogenic characterization of Colletotrichum sp. as a causative agent of anthracnose in yams Dioscorea sp.] Revista Colombiana de Biotecnologia 1: 24-35.
Plesset, J. L, Ludwing, J., Cox, B., and Mc Laughin, C. (1987) Effect of cell on thermotolerance in Saccharomyces cerevisiae. Journal of Bacteriology 169: 779-784.
Pusey, P. L. (1994). Enhancement of biological control agents for postharvest diseases and their integration with other control strategies. In: Wilson, C. L, Wisniewski, M. E. (Eds.), Biological Control of Postharvest Diseases. Theory and Practice. CRC Press, Boca Raton, W. Va., USA, pp. 77-88.
Ruiz, M., and Guadarrama, A. (1992). Comportamiento poscosecha del mango (Mangifera indica L.) tipo Bocado durante maduracion controlada. [Postharvest behavior of mango (Mangifera indica L.) Bocado type, during controlled ripening.] Revista de la Facultad de Agronomia, Maracay, Venezuela 18:79-93.
SAS (1998) User's Guide (Release 6.12) SAS Statistics Int'l Inc. Cary, N.C.
Seymour, G B, N'diaye, M., Wainwright, H., and Tucker, G. A. (1990). Effects of cultivar and harvest maturity on ripening of mangoes during storage. Journal of Horticultural Science 65:479-483.
Shanmuganathan N. (1996). Yeasts as a biological control for microbial diseases of fruit. U.S. Pat. No. 5,525,132.
Silva, J. Carvalho, A. S., Teixeira, P., and Gibas, P. A. (2002). Bacteriocin production by spray-dried lactic acid bacteria. Letters in Applied Microbiology 34: 77-81.
Smoot, J. J. Segall and R. H. (1963). Hot water as a postharvest control of mango anthracnose. Plant Disease Reporter 47: 739-742.
Spadaro, D. and Gullino, M. L. (2004) State of the art and future prospects of the biological control of postharvest fruit diseases. International Journal of Food Microbiology 91: 185-194.
Vega, P. A. (2001). Enfermedades del mango (Mangifera indica) [Mango diseases (Mangifera indica). In: Enfermedades y normatividad de Frutales del Pacifico Centro-Sur de Mexico [Diseases and regulations of fruit of the Pacific-South Center of Mexico] publishers: Fuentes and Ireta. pp. 49-61. Sociedad Mexicana de Fitopatologia A. C. Obregón, Sonora, Mexico.
Velankar, H. and Heble, M. (2003). Biotransformation of (L)-citronellal to (L)-citronellol by free and immobilized Rhodotorula minuta. Electronic Journal of Biotechnology 6 (2): 90-94.
Wan-Yin, F. and Mark, R. (1995). Spray drying of Lactococcus lactis ssp. lactis C2 and cellular injury. Journal of Food Science 60 (1): 195-200.
Werner-Washburn, M., Braun, E., Johnston, G. C, and Singer, R. A. (1993). Stationary phase in the yeast Saccharomyces cerevisiae. Microbiology Reviews 57 (2): 383-401.

1-6. (canceled)

7. A solid, dry composition for biological control of Colletotrichum gloeosporioides, the solid dry composition comprising viable cells of Rhodotorula minuta and having a refrigerated shelf life of at least 1 year.

8. The solid composition of claim 7, wherein the solid composition has a moisture content between 4.6 to 8.4%.

9. The solid composition of claim 7, wherein for at least one year the solid composition keeps a viable cell count of at least 1×10⁹ CFU/g.

10. The composition of claim 7, the composition further comprising a second biological control agent, the composition requiring lower doses of each biological control agent than would be required by the independent application of each to obtain similar levels of biological control.

11. The composition of claim 10, wherein the second biological control agent is Bacillus subtilis.

12. A method for biological control of disease caused by Colletotrichum gloeosporioides, the method comprising at least one pre-harvest application of effective doses of a solid, dry composition with Rhodotorula minuta effective for biological control of Colletotrichum gloeosporioides.

13. The method of claim 12, wherein the method of application is spraying the entire aerial part of the plant.

14. The method of claim 12, wherein the method controls anthracnose at levels equal to or higher than those obtained with treatment with a chemical fungicide.

15. The method of claim 12, wherein the solid dry composition is applied to a tropical or subtropical crop.

16. The method of claim 12, wherein the solid dry composition is applied to a crop selected from the group consisting of mango (Mangifera indica), papaya (Carica papaya L.), avocado (Persea americana), soursop (Annona muricata), mandarin (Citrus reticulata, C. unshiu and C. reshni), rough lemon (Citrus jambhiri Lush), strawberry (Fragaria ananassa L.), orchids (Orchidaceae) and yam (Dioscorea sp.).

17. The method of claim 16, wherein the crop is mango (Mangifera indica).

18. The method of claim 17, wherein the mango crop is c. v. Kent.

19. A method for reducing weight loss during storage of mango, the method comprising at least one pre-harvest application of a solid, dry composition, with Rhodotorula minuta, effective for biological control of Colletotrichum gloeosporioides.

20. A method for producing a solid, dry composition for the biological control of Colletotrichum gloeosporioides, the solid dry composition comprising Rhodotorula minuta, with a shelf life of at least 1 year, the method comprising:
(a) culturing in a submerged fashion Rhodotorula minuta in a suitable medium, with suitable parameters of temperature, stirring, aeration and pH for a sufficient time for the culture to reach a stationary stage; and
(b) drying the cell suspension at temperatures appropriate to maintain a suitable proportion of viable cells.

21. The method of claim 20, wherein step (a) produces a cell pack, and further comprising the following step after step (a):
(a)(1) recovering and resuspending the cell pack in an appropriate buffer.

22. The method of claim 21, further comprising the following step after step (a)(1):
(a)(2) adding a support and/or a thermoprotector to the cell suspension.

23. The method of claim 20, wherein values of main control parameters for the submerged culture of Rhodotorula minuta, are as follows:
pH: 4 to 9,
temperature: 30° C.,
stirring: 100 to 400 rpm,
aeration: 0.5 to 1.0 air volumes per medium volume per minute.

24. The method of claim 21, wherein the cell pack is recovered by centrifugation.

25. The method of claim 22, wherein the support comprises a flour with high starch content.

26. The method of claim 25, wherein the flour is cornstarch.

27. The method of claim 20, wherein the drying is carried out in a spray dryer.
